(19) United States
(12) Patent Application Publication
(10) Pub. No.: US 2006/0009816 A1
(43) Pub. Date: Jan. 12, 2006

(54) Percutaneous intramuscular stimulation system

(75) Inventors: Zi-Ping Fang, Mayfield Village, OH (US); Soheyl Pourmehdi, Beachwood, OH (US)

Correspondence Address: RYAN KROMHOLZ & MANION, S.C., POST OFFICE BOX 26618, MILWAUKEE, WI, 53226, US

(73) Assignee: NeuroControl Corporation

(21) Appl. No.: 11/228,084

(22) Filed: Sep. 16, 2005

Related U.S. Application Data
(63) Continuation of application No. 09/862,156, filed on May 21, 2001, now abandoned.
Application No. 09/862,156 is a continuation of application No. 09/089,994, filed on Jun. 3, 1998, now abandoned.

(51) Int. Cl. A61N 1/18

(52) U.S. Cl. 607/48

(57) ABSTRACT

A percutaneous, intramuscular stimulation system for therapeutic electrical stimulation of select muscles of a patient includes a plurality of intramuscular stimulation electrodes (50) for implantation directly into select muscles of a patient and an external battery-operated, microprocessor-based stimulation pulse train generator (10) for generating select electrical stimulation pulse train signals (T). A plurality of insulated electrode leads (40) percutaneously, electrically interconnect the plurality of intramuscular stimulation electrodes (50) to the external stimulation pulse train generator (10), respectively. The external pulse train generator (10) includes a plurality of electrical stimulation pulse train output channels (E) connected respectively to the plurality of percutaneous electrode leads (40) and input means (24,26,28) for operator selection of stimulation pulse train parameters (PA,PD,PF) for each of the stimulation pulse train output channels (E) independently of the other channels. Visual output means (20) provides visual output data to an operator of the pulse train generator (10). Non-volatile memory means (66,68) stores the stimulation pulse train parameters for each of the plurality of stimulation pulse train output channels (E). The generator (10) includes means for generating stimulation pulse train signals (100,102) with the selected pulse train parameters on each of the plurality of stimulation pulse train output channels (E) so that stimulus pulses of the pulse train signals having the select stimulation pulse train parameters pass between the intramuscular electrodes (50) respectively connected to the stimulation pulse train output channels (E) and a reference electrode (52).

### RELATED APPLICATIONS

This application is a continuation of co-pending U.S. patent application Ser. No. 09/862,156, filed May 21, 2001, which is a continuation of U.S. patent application Ser. No. 09/089,994, filed Jun. 3, 1998, now abandoned.

### BACKGROUND OF THE INVENTION

The present invention relates to the art of therapeutic neuromuscular stimulation. It finds particular application for use by human patients who are paralyzed or partially paralyzed due to cerebrovascular accidents such as stroke or the like. The invention is useful for retarding or preventing muscle disuse atrophy, maintaining extremity range-of-motion, facilitating voluntary motor function, relaxing spastic muscles, increasing blood flow to select muscles, and the like.

An estimated 555,000 persons are disabled each year in the United States of America by cerebrovascular accidents (CVA) such as stroke. Many of these patients are left with partial or complete paralysis of an extremity. For example, subluxation (incomplete dislocation) of the shoulder joint is a common occurrence and has been associated with chronic and debilitating pain among stroke survivors. In stroke survivors experiencing shoulder pain, motor recovery is frequently poor and rehabilitation is impaired. Thus, the patient may not achieve his/her maximum functional potential and independence. Therefore, prevention and treatment of subluxation in stroke patients is a priority.

There is a general acknowledgment by healthcare professionals of the need for improvement in the prevention and treatment of shoulder subluxation. Conventional intervention includes the use of orthotic devices, such as slings and supports, to immobilize the joint in an attempt to maintain normal anatomic alignment. The effectiveness of these orthotic devices varies with the individual. Also, many authorities consider the use of slings and arm supports to be controversial or even contraindicated because of the potential complications from immobilization including disuse atrophy and further disabling contractures.

Surface, i.e., transcutaneous, electrical muscular stimulation has been used therapeutically for the treatment of shoulder subluxation and associated pain, as well as for other therapeutic uses. Therapeutic transcutaneous stimulation has not been widely accepted in general because of stimulation-induced pain and discomfort, poor muscle selectivity, and difficulty in daily management of electrodes. In addition to these electrode-related problems, commercially available stimulators are relatively bulky, have high energy consumption, and use cumbersome connecting wires.

In light of the foregoing deficiencies, transcutaneous stimulation systems are typically limited to two stimulation output channels. The electrodes mounted on the surface of the patient's skin are not able to select muscles to be stimulated with sufficient particularity and are not suitable for stimulation of the deeper muscle tissue of the patient as required for effective therapy. Any attempt to use greater than two surface electrodes on a particular region of a patient's body is likely to result in suboptimal stimulation due to poor muscle selection. Further, transcutaneous muscle stimulation via surface electrodes commonly induces pain and discomfort.

Studies suggest that conventional interventions are not effective in preventing or reducing long term pain or disability. Therefore, it has been deemed desirable to develop a percutaneous, i.e., through the skin, neuromuscular stimulation system that utilizes temporarily implanted, intramuscular stimulation electrodes connected by percutaneous electrode leads to an external and portable pulse generator.

### SUMMARY OF THE INVENTION

In accordance with a first aspect of the present invention, a percutaneous, intramuscular stimulation system for therapeutic electrical stimulation of select muscles of a patient includes a plurality of intramuscular stimulation electrodes for implantation directly into selected muscles of a patient and an external battery-operated, microprocessor-based stimulation pulse train generator for generating select electrical stimulation pulse train signals. A plurality of insulated electrode leads are used for percutaneously interconnecting the plurality of intramuscular stimulation electrodes to the external stimulation pulse train generator, respectively. The external pulse train generator includes a plurality of electrical stimulation pulse train output channels connected respectively to the plurality of percutaneous electrode leads and input means for operator selection of stimulation pulse train parameters for each of the stimulation pulse train output channels independently of the other channels. The stimulation pulse train parameters include at least pulse amplitude and pulse width or duration for stimulation pulses of the stimulation pulse train, and an interpulse interval between successive pulses of the stimulation pulse train defining a pulse frequency. Visual output means provides visual output data to an operator of the pulse train generator. The visual output data includes at least the stimulation pulse train parameters for each of the stimulation pulse train output channels. Non-volatile memory means stores the stimulation pulse train parameters for each of the plurality of stimulation pulse train output channels. The generator includes means for generating stimulation pulse train signals with the selected pulse train parameters on each of the plurality of stimulation pulse train output channels so that stimulus pulses of the pulse train signals having the select stimulation pulse train parameters pass between the intramuscular electrodes respectively connected to the stimulation pulse train output channels and a reference electrode.

In accordance with another aspect of the invention, a method of stimulating select muscle tissue of a patient includes programming a patient external stimulation pulse generator with at least one stimulation pulse train session including at least one stimulation cycle defining a stimulation pulse train envelope for a plurality of stimulation pulse train output channels. Each envelope is defined by at least a ramp-up phase of a first select duration wherein pulses of a stimulus pulse train progressively increase in charge, a hold phase of a second select duration wherein pulses of the stimulus pulse train are substantially constant charge, and a ramp-down phase of a third select duration wherein pulses of the stimulus pulse train progressively decrease in charge. A plurality of intramuscular electrodes are implanted into select muscle tissue of the patient and electrically connected to the plurality of output channels, respectively, of the pulse train generator. On each of said plurality of stimulation output channels and in accordance with a respective envelope, stimulation pulse train signals are generated with the generator so that the select muscle tissue of the patient is stimulated in accordance with the at least one stimulation cycle.

One advantage of the present invention is the provision of a therapeutic percutaneous intramuscular stimulation system that retards or prevents muscle disuse atrophy, maintains muscle range-of-motion, facilitates voluntary motor function, relaxes spastic muscles, and increases blood flow in selected muscles.

Another advantage of the present invention is that it provides a therapeutic muscular stimulation system that uses intramuscular, rather than skin surface (transcutaneous) electrodes to effect muscle stimulation of select patient muscles.

Another advantage of the present invention is that it provides a small, lightweight, and portable battery-operated external pulse generator.

A further advantage of the present invention is that it avoids the use of skin surface electrodes which are inconvenient, not sufficiently selective to stimulate only particular muscles, require daily application by the patient, are subject to patient misapplication, and that have been found to cause pain or discomfort upon muscle stimulation.

Still another advantage of the present invention resides in the provision of a therapeutic stimulation system that allows for precise muscle selection through use of intramuscular electrodes, including stimulation of deep muscles not readily stimulated via transcutaneous stimulation techniques and associated surface mounted electrodes.

Yet another advantage of the present invention is that it is “user-friendly,” allowing selective variation of system operational parameters by a therapist or patient without the need for any external programming apparatus such as a personal computer or the like.

A further advantage of the present invention is the provision of a percutaneous stimulation system with low power consumption, long battery life (e.g., up to 50 hours of use)

A still further advantage of the present invention is the provision of a percutaneous, intramuscular stimulation system including a “hot-button” for selective instantaneous pulse train generation during system setup to facilitate adjustment of stimulation pulse train parameters during system setup.

A yet further advantage of the present invention is found in a percutaneous intramuscular stimulation system which logs patient usage for subsequent review by a doctor or therapist to ensure patient compliance with prescribed therapeutic stimulation routines.

The foregoing advantages and others will increase patient acceptance, reduce the service time required from clinicians, and prevent secondary patient injury requiring additional medical treatment.

Still further benefits and advantages of the present invention will become apparent to those of ordinary skill in the art upon reading and understanding the following detailed description of the preferred embodiments.

### BRIEF DESCRIPTION OF THE DRAWINGS

The invention may take form in various components and arrangements of components, and in various steps and arrangements of steps. The drawings are only for purposes of illustrating preferred embodiments, and are not to be construed as limiting the invention.

FIG. 1A is a front elevational view of a portable, programmable stimulation pulse train generator in accordance with the present invention;

FIGS. 1B-1D are top, bottom, and right-side elevational views of the stimulation pulse train generator of FIG. 1A;

FIG. 2 illustrates a preferred intramuscular electrode and percutaneous electrode lead;

FIG. 3 diagrammatically illustrates the structure and operation of the percutaneous intramuscular stimulation system in accordance with the present invention;

FIG. 3A diagrammatically illustrates a preferred pulse amplitude/duration controller, current driver, and impedance detector circuit in accordance with the present invention; and,

FIG. 4 graphically illustrates the stimulation paradigm of the percutaneous intramuscular stimulation system in accordance with the present invention.

### DETAILED DESCRIPTION OF THE PREFERRED EMBODIMENTS

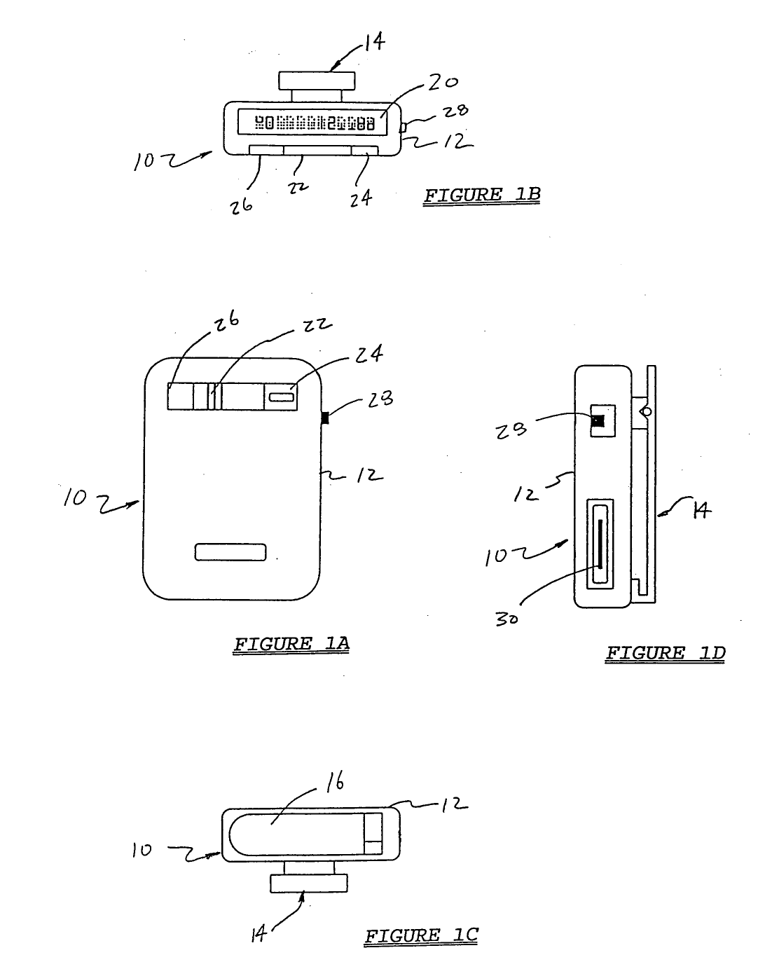

With reference to FIGS. 1A-1D, the percutaneous, intramuscular stimulation system in accordance with the present invention includes an electrical stimulation pulse generator 10. The pulse generator 10 includes a lightweight, durable plastic housing 12 fabricated from a suitable plastic or the like. The case 12 includes a clip 14 that allows the pulse generator 10 to be releasably connected to a patient's belt, other clothing, or any other convenient location. The case 12 also includes a releasable battery access cover 16.

For output of visual data to a patient or clinician operating the stimulation system, a visual display 20 is provided. The display 20 is preferably provided by a liquid crystal display, but any other suitable display means may alternatively be used. An audio output device, such as a beeper 22 is also provided.

For user control, adjustment, and selection of operational parameters, the stimulation pulse generator 10 includes means for input of data. Preferably, the pulse generator 10 includes an increment switch 24, a decrement switch 26, and a select or “enter” switch 28. The increment and decrement switches 24, 26 are used to cycle through operational modes or patterns and stimulation parameters displayed on the display 20, while the select switch 28 is used to select a particular displayed operational pattern or stimulation parameter. The select switch 28 also acts as a power on/off toggle switch. By choosing the appropriate mode, the select switch 28 can be selectively armed as a “hot button.” During adjustment of stimulation pulse train parameters, a clinician is able to activate the hot button to test, instantaneously, the effect of the selected stimulation pulse train parameters on the patient's muscles. This facilitates the quick and proper adjustment of the stimulation pulse train parameters without requiring the clinician to exit the setup procedure menu of the stimulation pulse generator 10.

For output of electrical stimulation pulse train signals, the pulse train generator 10 includes an external connection socket 30 that mates with a connector of an electrode cable assembly (not shown) to interconnect the pulse generator 10 with a plurality of intramuscular electrodes via percutaneous electrode leads. More particularly, the cable assembly connected to the socket 30 includes a second connector on a distal end that mates with a connector attached to the proximal end of each of the percutaneous stimulation electrode leads and a reference electrode lead.

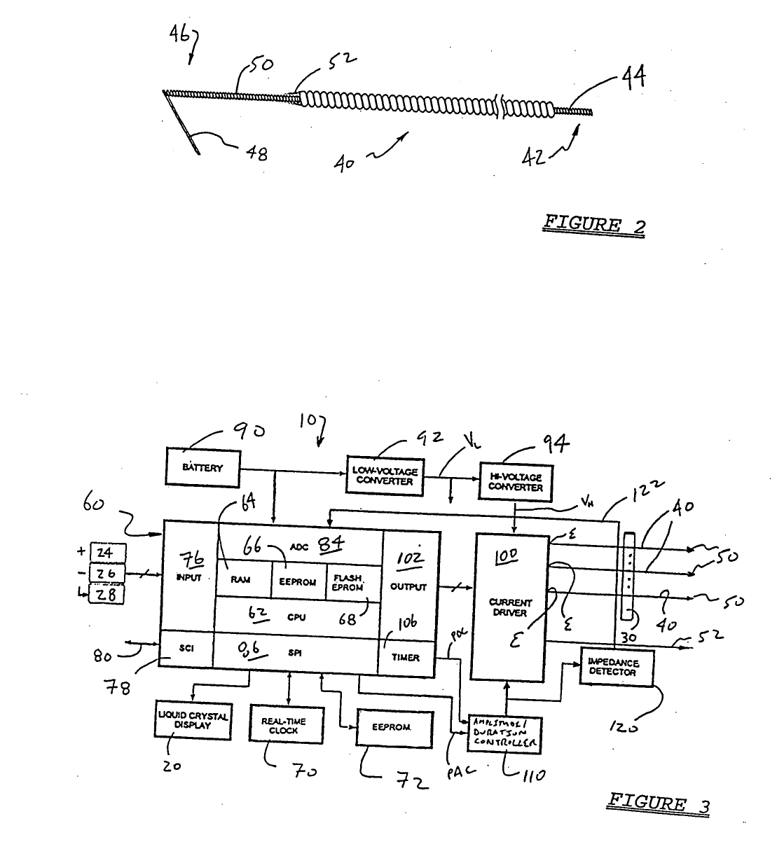

A preferred intramuscular electrode and percutaneous lead are shown in FIG. 2. The electrode lead 40 is fabricated from a 7-strand stainless steel wire insulated with a biocompatible polymer. Each individual wire strand has a diameter of 34 μm and the insulated multi-strand lead wire has a diameter of 250 μm. The insulated wire is formed into a spiral or helix as has been found preferred to accommodate high dynamic stress upon muscle flexion and extension, while simultaneously retaining low susceptibility to fatigue. The outer diameter of the helically formed electrode lead 40 is approximately 580 μm and it may be encased or filled with silicone or the like.

As mentioned above, a proximal end 44 of each of the plurality of intramuscular electrode lead wires 40 is located exterior to the patient's body when in use. The proximal end 44 includes a deinsulated length for connection to an electrical connector in combination with the remainder of the electrode leads. The distal end 46 of each lead 40, which is inserted directly into muscle tissue, also includes a deinsulated length which acts as the stimulation electrode 50. It is preferred that at least a portion of the deinsulated length be bent or otherwise deformed into a barb 48 to anchor the electrode in the selected muscle tissue. A taper 52, made from silicone adhesive or the like, is formed between the deinsulated distal end 50 and the insulated portion of the lead 40 to reduce stress concentration.

Unlike surface electrodes which are applied to the surface of the patient's skin using an adhesive, each of the plurality of percutaneous electrodes 50 is surgically implanted into select patient muscle tissue, and the associated electrode lead 40 exits the patient percutaneously, i.e., through the skin, for connection to the stimulation pulse generator 10. Preferably, each of the electrodes 50 is implanted into the select muscles by use of a hypodermic needle. Once all of the electrodes are implanted as desired, their proximal ends are crimped into a common connector that mates with the cable assembly which is, in turn, connected to the pulse generator 10 through the connection socket 30.

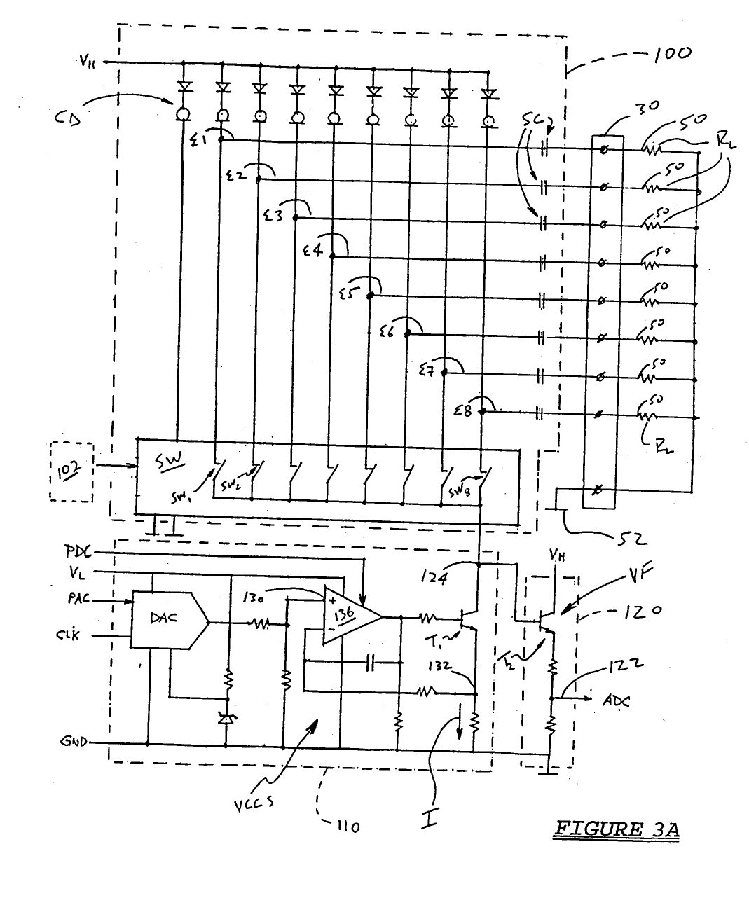

FIG. 3 diagrammatically illustrates the overall percutaneous, intramuscular stimulation system in accordance with the present invention. Unlike surface stimulation systems which exhibit poor muscle selectivity and are, thus, typically limited to two stimulation electrodes and channels, the present percutaneous, intramuscular stimulation system allows for precise muscle selection and use of three or more stimulation electrodes and channels. The preferred system in accordance with the present invention uses up to eight or more intramuscular electrodes 50, each connected to an independent electrode stimulation channel E, and a single reference electrode 52 which may be either an intramuscular or surface electrode. Those of ordinary skill in the art will also recognize that the use of intramuscular electrodes allows for selection and stimulation of deep muscle tissue not practicable by surface stimulation.

The stimulation pulse generator 10 comprises a microprocessor-based stimulation pulse generator circuit 60. The preferred microcontroller is a Motorola GSHC12, although other suitable microcontrollers may be used without departing from the scope and intent of the invention. The circuit 60 comprises a central processing unit (CPU) 62 for performing all necessary operations. Random access memory (RAM) 64 is present for temporary storage of operational data as needed by the CPU 62. A first nonvolatile memory means, such as electrically erasable programmable read only memory (EEPROM) 66, provides nonvolatile storage as needed for operational instructions or other information, although the first nonvolatile memory means may not necessarily be used. Preferably, flash EPROM 68 (rather than write-once EPROM) is provided for storage of software operating instructions. Use of flash EPROM 68 facilitates periodic, unlimited upgrade of the software operating instructions.

In order to log or record patient usage of the stimulation pulse generator 10, the stimulation circuit 60 includes a real-time clock 70 along with a second nonvolatile memory means such as EEPROM 72 to provide sufficient nonvolatile storage for recording and time-stamping a patient's use of the system. A clinician is thereafter able to access the EEPROM 72 to review the patient's use of the system to ensure patient compliance with the prescribed therapeutic stimulation protocol. Preferably, the second nonvolatile memory 72 also provides storage for all patient-specific stimulation protocols.

The increment, decrement, and select user input switches 24,26,28 are operatively connected into the circuit 60 via an input stage 76. In addition, a serial communication interface (SCI) 78 provides means for selectively connecting an external device, such as a computer, as needed by way of an RS-232 connection 80 or the like for data upload and download. An analog-to-digital converter 84 performs all analog-to-digital conversion of data as needed for processing in the circuit 60. A serial peripheral interface (SPI) 86 provides means for connecting peripheral components, such as the display 20, the clock 70, the EEPROM 72, and other components to the microcontroller.

Electrical potential or energy is supplied to the circuit 60 by a battery 90, preferably AA in size and ranging from 1.0-1.6 volts. A low-voltage dc-dc converter 92 adjusts the voltage supplied by the battery 90 to a select level V_{L}, preferably 3.3 volts. To minimize depletion of the battery during periods of inactivity of the pulse generator 10, the circuit 60 is programmed to automatically power-down after a select duration of inactivity. Those skilled in the art will recognize that the RAN 64 provides volatile storage, and the storage means 66,68,72 provide nonvolatile storage to prevent loss of data upon interruption of power to the circuit 60 through malfunction, battery depletion, or the like.

The output V_{L} of the low-voltage dc-dc converter 92 is also supplied to a high-voltage dc-dc converter 94 which steps-up the voltage to at least 30 volts. The high-voltage output V_{H}, of the dc-dc converter 94 provides the electrical potential for the stimulation pulse train signals transmitted to the plurality of intramuscular electrodes 50 through a current driver 100. More particularly, an output means 102 of the circuit 60 provides channel selection input to the current driver 100 to control the transmission of the high-voltage electrical potential from the driver 100 to the selected electrodes 50 on a selected one of the plurality of stimulation output channels E. Although only three output channels E are illustrated, those skilled in the art will recognize that a greater number of output channels may be provided. Preferably, eight output channels E are provided.

The electrical current passes between the selected electrodes 50 and the reference electrode 52. A pulse duration timer 106 provides timing input PDC as determined by the CPU 62 to the pulse amplitude/duration controller 110 to control the duration of each stimulation pulse. Likewise, the CPU 62 provides a pulse amplitude control signal PAC to the circuit 110 by way of the serial peripheral interface 86 to control the amplitude of each stimulation pulse. One suitable circuit means for output of stimulation pulses as described above is in accordance with that described in U.S. Pat. No. 5,167,229, the disclosure of which is hereby expressly incorporated by reference.

In order to ensure that an electrode lead is properly transmitting the stimulation pulse train signals to the select muscle tissue, an impedance detection circuit 120 monitors the impedance of each electrode lead 40. The impedance detection circuit 120 provides an analog impedance feedback signal 122 to the analog-to-digital converter 84 where it is converted into digital data for input to the CPU 62. Upon breakage of a lead 40 or other malfunction, the impedance detection circuit detects a change in impedance, and correspondingly changes the impedance feedback signal 122. The impedance feedback signal 122 allows the microcontroller to interrupt stimulation and/or generate and error signal to a patient or clinician.

FIG. 3A is a somewhat simplified diagrammatic illustration of a most preferred current driver circuit 100, pulse amplitude/duration control circuit 110, and impedance detection circuit 120. The illustrated current driver circuit 100 implements eight output channels E1-E8, each of which is connected to an electrode 50 implanted in muscle tissue for passing electrical current through the muscle tissue in conjunction with the reference electrode 52. Accordingly, the patient muscle tissue and implanted electrodes 50 are illustrated as a load R_{L} connected to each channel E1-E8.

Each output channel E1-E8 includes independent electrical charge storage means such as a capacitor SC which is charged to the high voltage V_{H} through a respective current limiting diode CD. To generate a stimulation pulse, the microcontroller output circuit 102 provides channel select input data to switch means SW, such as an integrated circuit analog switch component, as to the particular channel E1-E8 on which the pulse is to be passed. Switch means SW closes the selected switch SW₁-SW₈ accordingly. The microcontroller also provides a pulse amplitude control signal PAC into a voltage-controlled current source VCCS. The pulse amplitude control signal PAC is converted into an analog signal at 130 by the digital-to-analog converter DAC. The analog signal at 130 is supplied to an operational amplifier 136 which, in conjunction with the transistor T₁, provides a constant current output I from the voltage-controlled current source VCCS. Of course, those of ordinary skill in the art will recognize that the particular magnitude of the constant current I is varied depending upon the magnitude of the voltage signal at 130 input to the OP-AMP 136, i.e., the circuit VCCS is provided such that the voltage at point 132 seeks the magnitude of the voltage at point 130. As such, the pulse amplitude control signal PAC controls the magnitude of the current I, and the circuit VCCS ensures that the current I is constant at that select level as dictated by the pulse amplitude control input PAC. For stimulation of human muscle, it is preferable that the current I be within an approximate range of 1 mA-20 mA.

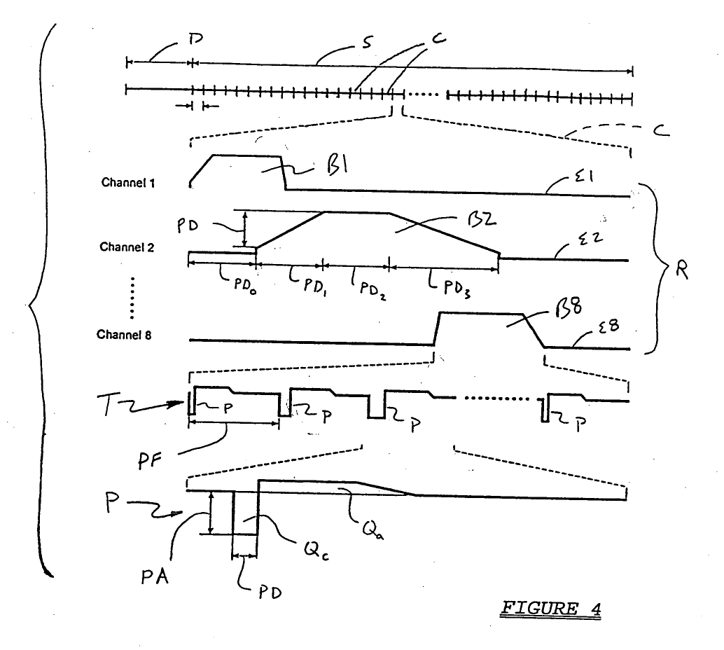

Upon closing one of switches SW₁-SW₈, the relevant capacitor SC discharges and induces the current I as controlled by the pulse amplitude control signal PAC and a pulse duration control signal PDC. The constant current I passes between the reference electrode 52 and the relevant one of the electrodes 50 to provide a cathodic pulse phase Q_{c} (see FIG. 4). The pulse duration PD of the phase Q_{c} is controlled by the microcontroller through a pulse duration control signal PDC output by the timer circuit 106 into the pulse amplitude/duration control circuit 110. In particular, the pulse duration control signal PDC is input to a shut-down input of the OP-AMP 136 to selectively enable or blank the output of the OP-AMP 136 as desired, and, thus, allow or stop the flow of current I between the electrodes 50,52.

Upon completion of the cathodic phase Q_{c} as controlled by the pulse duration control signal PDC, the discharged capacitor SC recharges upon opening of the formerly closed one of the switches SW₁-SW₈. The flow of recharging current to the capacitor SC results in a reverse current flow between the relevant electrode 50 and the reference electrode 52, thus defining an anodic pulse phase Qₐ. The current amplitude in the anodic pulse phase Qₐ is limited, preferably to 0.5 mA, by the current limiting diodes CD. Of course, the duration of the anodic phase is determined by the charging time of the capacitor SC, and current flow is blocked upon the capacitor becoming fully charged. It should be recognized that the interval between successive pulses or pulse frequency PF is controlled by the CPU 62 directly through output of the channel select, pulse amplitude, and pulse duration control signals as described at a desired frequency PF.

The impedance detection circuit 120 “detects” the voltage on the active channel E1-E8 (i.e., the channel on which a pulse is being passed) through implementation of a high-impedance voltage follower circuit VF using a transistor T₂. Accordingly, it will be recognized that the voltage at points 122 and 124 will move together. Accordingly, for example, in the event of breakage of an electrode lead 40, a drop in voltage at point 124 will cause a corresponding drop in voltage at point 122. The voltage signal at point 122 is fed back to the microcontroller analog-to-digital converter 84 for interpretation by the CPU 62 in accordance with stored expected values indicating preferred impedance ranges. At the same time, the CPU 62 knows which switch SW₁-SW₈ is closed. Therefore, the CPU 62 is able to determine the channel E1-E8 on which the lead breakage occurred.

The preferred stimulus pulse train paradigm is graphically illustrated in FIG. 4. A preferred design implements up to 4 independent preprogrammed patterns. For each pattern, a stimulation session S is pre-programmed into the stimulator circuit 60 by a clinician through use of the input means 24,26,28. Each session S has a maximum session duration of approximately 9 hours, and a session starting delay D. The maximum session starting delay D is approximately 1 hour. The session starting delay D allows a patient to select automatic stimulation session start at some future time. Within each session S, a plurality of stimulation cycles C are programmed for stimulation of selected muscles. Preferably, each stimulation cycle ranges from 2-100 seconds in duration.

With continuing reference to FIG. 4, a stimulus pulse train T includes a plurality of successive stimulus pulses P. As is described above and in the aforementioned U.S. Pat. No. 5,167,229, each stimulus pulse p is current-regulated and biphasic, i.e., comprises a cathodic charge phase Q_{c} and an anodic charge phase Qₐ. The magnitude of the cathodic charge phase Q_{c} is equal to the magnitude of the anodic charge phase Qₐ. The current-regulated, biphasic pulses P provide for consistent muscle recruitment along with minimal tissue damage and electrode corrosion.

Each pulse P is defined by an adjustable pulse amplitude PA and an adjustable pulse duration PD. The pulse frequency PF is also adjustable. Further, the pulse amplitude PA, pulse duration PD, and pulse frequency PF are independently adjustable for each stimulation channel E. The amplitude of the anodic charge phase Qₐ is preferably fixed at 0.5 mA, but may be adjusted if desired.

Pulse “ramping” is used at the beginning and end of each stimulation pulse train T to generate smooth muscle contraction. Ramping is defined herein as the gradual change in cathodic pulse charge magnitude by varying at least one of the pulse amplitude PA and pulse duration PD. In FIG. 4, the preferred ramping configuration is illustrated in greater detail. As mentioned, each of the plurality of stimulation leads/electrodes 40,50 is connected to the pulse generator circuit 60 via a stimulation pulse channel E. As illustrated in FIG. 4, eight stimulation pulse channels E1,E2,E8 are provided to independently drive up to eight intramuscular electrodes 50. Stimulation pulse trains transmitted on each channel E1-E8 are transmitted within or in accordance with a stimulation pulse train envelope B1-B8, respectively. The characteristics of each envelope B1-B8 are independently adjustable by a clinician for each channel E1-E8. Referring particularly to the envelope B2 for the channel E2, each envelope B1-B8 is defined by a delay or “off” phase PD₀ where no pulses are delivered to the electrode connected to the subject channel, i.e., the pulses have a pulse duration PD of 0. Thereafter, according to the parameters programmed into the circuit 60 by a clinician, the pulse duration PD of each pulse P is increased or “ramped-up” over time during a “ramp-up” phase PD₁ from a minimum initial value (e.g., 5 μsec) to a programmed maximum value. In a pulse duration “hold” phase PD₂, the pulse duration PD remains constant at the maximum programmed value. Finally, during a pulse duration “ramp-down” phase PD₃, the pulse duration PD of each pulse P is decreased over time to lessen the charge delivered to the electrode 50.

This “ramping up” and “ramping down” is illustrated even further with reference to the stimulation pulse train T which is provided in correspondence with the envelope ES of the channel ES. In accordance with the envelope B8, the pulses P of the pulse train T first gradually increase in pulse duration PD, then maintain the maximum pulse duration PD for a select duration, and finally gradually decrease in pulse duration PD.

As mentioned, the pulse amplitude PA, pulse duration PD, pulse frequency PF, and envelope P1-PS are user-adjustable for every stimulation channel E, independently of the other channels. Preferably, the stimulation pulse generator circuit 60 is pre-programmed with up to four stimulation patterns which will allow a patient to select the prescribed one of the patterns as required during therapy.

Most preferably, the pulse generator 10 includes at least up to eight stimulation pulse channels E. The stimulation pulse trains T of each channel E are sequentially or substantially simultaneously transmitted to their respective electrodes 50. The pulse frequency PF is preferably adjustable within the range of approximately 5 Hz to approximately 50 Hz; the cathodic amplitude PA is preferably adjustable within the range of approximately 1 mA to approximately 20 mA; and, the pulse duration PD is preferably adjustable in the range of approximately 5 μsec to approximately 200 μsec, for a maximum of approximately 250 pulses per second delivered by the circuit 60.

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

Having thus described the preferred embodiments, the invention is now claimed to be:

1. A percutaneous, intramuscular stimulation system for therapeutic electrical stimulation of select muscles of a patient, said stimulation system comprising:
a plurality of intramuscular stimulation electrodes for implantation directly into selected muscles of a patient, each electrode including an insulated percutaneous lead;
an external battery-operated, microprocessor-based stimulation pulse train generator for generating select electrical stimulation pulse trains, said external pulse train generator including:
a plurality of electrical stimulation pulse train output channels connected respectively to said plurality of percutaneous electrode leads;
an input device for operator selection of stimulation pulse train parameters for each of said stimulation pulse train output channels independently of the other channels, said stimulation pulse train parameters including at least a pulse amplitude and pulse duration for stimulation pulses of said stimulation pulse train, and an interpulse interval between successive pulses of said stimulation pulse train defining a pulse frequency;
a visual output display which provides visual output data to an operator of the pulse train generator, said visual output data including at least said stimulation pulse train parameters for each of said stimulation pulse train output channels;
a non-volatile memory which stores said stimulation pulse train parameters for each of said plurality of stimulation pulse train output channels; and,
a pulse train generation system for generating stimulation pulse train signals with the select pulse train parameters on each of said plurality of stimulation pulse train output channels so that stimulus pulses of said pulse train signals having the select stimulation pulse train parameters pass between the intramuscular electrodes respectively connected to said stimulation pulse train output channels and a reference electrode.
